(19) United States
(12) Patent Application Publication
(10) Pub. No.: US 2026/0194457 A1
(43) Pub. Date: Jul. 9, 2026

(54) MEASURING METHOD FOR DETERMINING A PROTECTION FACTOR, AND PROTECTION FACTOR EVALUATION SYSTEM

(71) Applicant: COURAGE + KHAZAKA ELECTRONIC GMBH, Koln (DE)

(72) Inventors: Georg KHAZAKA, Koln (DE); Carina REBLE, Koln (DE); Diana KHAZAKA-OHMANN, Koln (DE)

(21) Appl. No.: 19/131,519

(22) PCT Filed: Nov. 16, 2023
(86) PCT No.: PCT/EP2023/082083

§ 371 Date: May 20, 2025

(30) Foreign Application Priority Data
Nov. 21, 2022 (DE) 10 2022 130 758.1

(51) Int. Cl. G01N 21/31; G01N 33/15

(52) U.S. Cl. CPC G01N 21/31; G01N 33/15

(57) ABSTRACT

The invention describes a method for determining a protection factor, comprising a spectroscopic measurement with the method steps of emitting radiation from a beam source, with the emitted radiation comprising light at an irradiation wavelength of between 280 nm and 2000 nm, irradiating a measuring body using the emitted radiation, detecting the radiation diffusely reflected by the irradiated measuring body at a detection wavelength, evaluating the protection factor of a protection means at an evaluation wavelength from the diffusely reflected radiation and a transmission spectrum, with the data of a transmission spectrum being used for the evaluation of the protection factor of the protection means, with the evaluation wavelength differing from the wavelength range of the irradiation wavelength and/or the detection wavelength, and with the data of the transmission spectrum being in silico data. The invention also describes an SPF evaluation system for examining the protection factor of protection means, comprising the following components: a measuring device having a beam source device, the beam source device comprising a beam source, a detector unit, a control unit for controlling the measuring device, and an evaluation unit.

The invention relates to a method for determining a protection factor, comprising the method steps of emitting radiation from a beam source, with the emitted radiation comprising light at an irradiation wavelength of between 280 nm and 2000 nm, irradiating a measuring body using the emitted radiation, detecting the radiation diffusely reflected by the irradiated measuring body at a detection wavelength, evaluating the protection factor of a protection means at an evaluation wavelength from the diffusely reflected radiation and a transmission spectrum, with the data of a transmission spectrum being used for the evaluation of the protection factor of the protection means, with the evaluation wavelength differing from the wavelength range of the irradiation wavelength and/or the detection wavelength, and with the data of the transmission spectrum being in silico data. The invention also describes an SPF evaluation system for examining the protection factor of protection means, comprising the following components: a measuring device having a beam source device, the beam source device comprising a beam source, a detector unit, a control unit for controlling the measuring device, and an evaluation unit.

### PRIOR ART

Protection The methods previously approved by the authorities of the European Union (EU) and the American Food and Drug Administration (FDA) for determining the SPF (Sun Protect Factor) are all harmful to the participating test subjects by causing erythema, i.e. a light-induced inflammatory reaction of the skin (COLI PA-15 European Cosmetic, Toiletry and Perfumery Association: Colipa SPF Test Method 94/289, 1994; ISO standards 24442, 24443, 24444). Therefore, both the FDA and the EU have repeatedly pointed out that future research activities must be directed towards new methods for characterizing the protective efficacy of sunscreen products in order to avoid long-term effects for the test subjects (European Commission, 20 Standardisation Mandate Assigned To CEN Concerning Methods For Testing Efficacy Of Sunscreen Products, M/389 EN, Brussels, 12 Jul. 2006).

This invention is intended to achieve this object. The existing procedures are defined in various sources:

### Procedures Defined in Standards and Regulations:

a. ISO 24444 defines a method for the in vivo determination of the SPF. The method is based on the generation of erythema on the skin of test subjects by radiation in the UVB range. Therefore, the method is harmful to the test subject.
b. ISO 24443 defines an in vitro method for the determination of the UVA protection factor (UVAPF). The protection means is applied to a plastic plate so that a transmission spectrum of the protection means can be measured. Due to uncontrollable fluctuations in the procedure, the transmission spectrum is adapted to the result of the erythema test according to ISO 24444 by scaling and is therefore dependent on its implementation. The plastic plate used has a roughened surface and is an unrealistic skin model.
c. ISO 24442 defines an in vivo method in which the UVA protection factor is determined using the minimum UVA dose required to produce irreversible pigmentation (suntan) of the skin. This method also causes a change in the test subject's skin.

### Patented Methods:

DE 198 28 497 A1 describes a method in which, as in ISO 24444, erythemas are induced in test subjects by UV irradiation of the skin. In contrast to ISO 24444, the erythemas are detected by reflection spectroscopy. The method is therefore also damaging. The optical effect (protection) of the protection means is not recorded by direct optical measurements, but by a biological reaction of the body.

DE 10 2004 020 644 A1 describes a method in which the generation of radicals by UV exposure is quantitatively measured in vivo using electron spin resonance (ESR). Here, too, the optical effect of the protection means is only recorded indirectly. In addition, measuring ESR is technically complex and requires relatively large, stationary devices (tabletop devices). They are also sensitive to interference from high-frequency radiation or rapid temporary magnetic field changes, such as those caused by electrical switching processes.

To determine the label SPF of topically applied protection means in vivo, test methods such as ISO 24444, the FDA Guideline or the Australian Standard are used worldwide. The basis of all these methods is the induction of an erythemal skin reaction by irradiating the skin with UV light. This is necessary to determine the minimum erythemal dose of untreated (MEDu) and product-treated skin (MEDp). Reliable in vitro methods in which human skin is replaced by synthetic substrate carriers are not available for SPF determination.

Monochromatic devices are known that use conventional xenon lamps and are therefore expensive to purchase and operate. Built-in monochromators measure different wavelengths one after the other, which is disadvantageous when the test subjects are moving. Polychromatic devices also use xenon lamps. The in vivo measured value is weighted using a filter so that it matches the in vivo UVA PF. Multi-LED devices for testing institutes represent another variant, which, however, is significantly larger and more expensive due to the spectroscopic detection.

It is therefore an object of the invention to provide a method for determining a protection factor which reduces the exposure to radiation on the human skin, which provides high-quality analysis results and at the same time is quick and easy to carry out.

It is also an object of the invention to provide an SPF evaluation system for the assessment of the protection factor of protection means, which provides high-quality analysis results, reduces the exposure to radiation on the human skin and is cost-effective to manufacture and operate.

The object is achieved by the method according to the invention for determining a protection factor. Advantageous embodiments of the invention are set out in the dependent claims below.

The method according to the invention for determining a protection factor comprising a spectroscopic measurement has four method steps: In the first method step, radiation is emitted from a beam source, wherein the emitted radiation comprises light at an irradiation wavelength between 280 nm and 2000 nm, preferably 280 to 800 nm, particularly preferably the range from 280 to 500 nm. In the context of this document, a beam source is a technical device for generating electromagnetic radiation. A beam source is therefore not an optical element for guiding, deflecting or changing the intensity and/or wavelength of electromagnetic radiation. A beam source is therefore not, for example, a light guide, grating, prism, or filter. The beam source generates electromagnetic radiation at an irradiation wavelength between the blue spectral range (from approximately 400 nm to 500 nm wavelength) and the UV range (280 nm to 400 nm).

The wavelength ranges are defined as follows:
<320 nm (UVB) with <0.1% of the total UV intensity,
320 nm to 340 nm (UVA II) 8% to 20% of the total UVA intensity
340 nm to 400 nm (UVA I) 80% to 92% of the total UVA intensity
400 nm to 500 nm, blue light

Preferably, the irradiation wavelength covers a wavelength range between 280 nm and 500 nm.

In the second method step, a measuring body is irradiated with the emitted radiation. In the case of an in vivo measurement, the measuring body is the human skin covered with the protection means to be tested; in the case of an in vitro measurement, it is a standardized test body covered with the protection means to be tested.

In the third method step, the diffusely reflected radiation of the irradiated measuring body is detected at a detection wavelength. The ratio of the intensities of the diffusely reflected radiation to the radiation coupled into the measuring body is a measure of the protective capability of the protection means. The detection wavelength, like the irradiation wavelength, preferably comprises a wavelength range, wherein the wavelength range of the detection wavelength preferably lies within the range of the irradiation wavelength or comprises the entire range of the irradiation wavelength. The spectroscopic measurement therefore includes in particular the recording of the diffusely reflected spectrum I(λ) (diffuse backscattering).

In the fourth step of the method, the protection factor of a protection means is evaluated using an evaluation wavelength from the diffusely reflected radiation and a transmission spectrum, wherein the data of a transmission spectrum are used to evaluate the protection factor of the protection means. A transmission spectrum is determined based on the UV transmittance of protective films in vitro. The substrates to which the protective films are applied only approximately replicate the inhomogeneous surface structure of human skin, such as, for example, polymethyl methacrylate (PMMA) sheets with a rough surface according to ISO 24443. The transmission spectrum data include intensity versus wavelength in preferably digitized format.

The evaluation wavelength is the wavelength for which the protection factor is determined. The evaluation wavelength is different from the wavelength range of the irradiation wavelength and/or the detection wavelength. The evaluation wavelength, like the irradiation wavelength and the detection wavelength, is preferably a wavelength range, wherein the wavelength range of the evaluation wavelength comprises at least the wavelength range of the irradiation wavelength and/or the detection wavelength.

Advantageously, the transmission spectrum data are in silico data. The data of the transmission spectrum are therefore neither determined in vivo on a test subject nor in vitro according to ISO 24443, but are estimated or determined mathematically. If the properties of the filter substances of a protection means are known, the transmission can be calculated and simulated. Based on the simulated transmission, the sun protection factor and all parameters that characterize the protection factor can be calculated.

The protection factor (SPF) is a scientific measure and indicates how much lower the risk of skin damage is when using a protection means. This factor focuses on the time it takes for UVB rays to penetrate a protection means and cause the skin to turn red (minimal erythema, MED) compared to the time it takes when no protection means is present. The dose of solar radiation required to cause skin reddening is divided by the dose required to cause reddening without protection means. This calculation is based on the application of 2 milligrams of protection means per square centimeter of skin surface. Currently, the SPF of protection means is determined by in vivo irradiation using a sun simulator (ISO 24444:2010 “Cosmetics—Sun protection test methods—In vivo determination of the sun protection factor (SPF)”), which represents the current state of the art. The basis for current testing of protection means is that test subjects are irradiated before and after the application of protection means.

The advantage of the method according to the invention lies in particular also in the fact that the in silico data do not represent a norm and can currently only be used for estimation. However, the deviation of the estimate from the actual data is significantly improved with the inventive method.

In a further development of the invention, the wavelength range of the transmission spectrum comprises the evaluation wavelength. The evaluation of the protection factor of the protection means is based on the in vivo diffusely reflected radiation and the in silico transmission spectrum. Due to its high absorption properties, human skin does not emit enough UVB radiation to measure the absorption spectrum of the applied product in the UVB range. It is therefore necessary to record the absorption spectrum of the test material in the UVB part of the spectrum (280-320 nm) separately using a different technique. The approach applied in this document uses the in vivo evaluation of the absolute UVA absorption spectrum as measured by an in vivo measurement with the use of a calculated in silico transmission spectrum to determine the protection factor of the protection means for a user. The evaluation and hybridization of an in vivo remission spectrum with an in silico transmission spectrum is carried out to obtain a complete UV spectrum, so that the protection factors are calculated according to the formulas of the applicable standard (ISO 24443). For this purpose, the wavelength range of the transmission spectrum includes the evaluation wavelength, which preferably covers the range from 280 nm to 500 nm.

In a further embodiment of the invention, the evaluation wavelength comprises the wavelength range from 280 nm to 2000 nm, preferably the wavelength range from 280 nm to 800 nm or particularly preferably the wavelength range from 280 nm to 500 nm. In a further development, the evaluation wavelength covers a wavelength range from 400 nm to 500 nm and preferably from 400 nm to 450 nm. The wavelength range of blue light, which borders on the UVA range (up to 400 nm), is preferably evaluated. To determine the protective ability of the protection means in the UVB wavelength range (<320 nm), this wavelength range can also be optionally evaluated.

In a further embodiment of the invention, the evaluation of the protective ability of the protection means for light in a wavelength range from 400 nm to 500 nm is carried out in a separate process from the evaluation of the protective ability of the protection means for light in a wavelength range from 280 nm to 400 nm. Due to its high absorption properties, human skin does not emit enough UVB radiation to measure the absorption spectrum of the applied product in the UVB range. It is therefore necessary to record the absorption spectrum of the test material in the UVB part of the spectrum (280-320 nm) separately using a different technique. For this purpose, the in vivo remission spectrum in the wavelength range from 320 nm to 400 nm, the wavelength range from 280 nm to 320 nm, and the wavelength range from 400 nm to 500 nm are recorded with an in silico transmission spectrum.

In a further embodiment of the invention, the evaluation of the protective ability of the protection means for light in a wavelength range from 400 nm to 500 nm is carried out in a different process from the evaluation of the protective ability of the protection means for light in a wavelength range from 280 nm to 400 nm. Due to its high absorption properties, human skin does not emit enough UVB radiation to measure the absorption spectrum of the applied product in the UVB range. It is therefore necessary to record the absorption spectrum of the test material in the UVB part of the spectrum (280-320 nm) separately using a different technique. For this purpose, the in vivo remission spectrum in the wavelength range from 320 nm to 400 nm, the wavelength range from 280 nm to 320 nm and the wavelength range from 400 nm to 500 nm is recorded with an in silico transmission spectrum.

In a further embodiment of the invention, the wavelength range of the irradiation wavelength or the wavelength range of the detection wavelength is smaller than the wavelength range of the evaluation wavelength. The wavelength ranges of the irradiation wavelength and the detection wavelength are preferably the same in a wavelength range of maximum 320 nm to 400 nm. The wavelength range of the evaluation wavelength covers a maximum range of 280 nm to 500 nm.

In a further embodiment of the invention, the wavelength range of the irradiation wavelength or the wavelength range of the detection wavelength in the wavelength range of the evaluation wavelength is smaller than the wavelength range of the evaluation wavelength. The wavelength ranges of the irradiation wavelength and the detection wavelength are preferably equal in a wavelength range of 320 nm to 400 nm, wherein wavelength ranges of the irradiation wavelength and the detection wavelength are smaller than said wavelength range of 320 nm to 400 nm. In the wavelength range of the evaluation wavelength (maximum 280 nm to 500 nm), the wavelength range of the irradiation wavelength or the wavelength range of the detection wavelength in the wavelength range of the evaluation wavelength is then smaller than the wavelength range of the evaluation wavelength.

In a further development of the invention, the wavelength range of the irradiation wavelength and/or the wavelength range of the detection wavelength is less than 100 nm, preferably less than 50 nm and particularly preferably less than 25 nm. Therefore, only one steel source is needed to emit electromagnetic radiation, which has a narrow wavelength range of the irradiation wavelength. Similarly, to record the remission spectrum, a detector is required that can detect a small wavelength range of the detection wavelength. The beam source and detector can therefore be designed to be cost-effective in both manufacture and operation.

In a further embodiment of the invention, the irradiation wavelength and/or the detection wavelength comprises only light with wavelengths outside the wavelength range of 400 nm to 450 nm. To record an in vivo measurement, the UVA wavelength range (320 nm-400 nm) in particular is coupled into the measuring body.

In a further embodiment of the invention, the irradiation wavelength and/or the detection wavelength comprises only light with wavelengths outside the wavelength range of 400 nm to 500 nm. In particular, the wavelength range of UVA and UVB is irradiated. This wavelength range poses the greatest risk to human skin, thus determining the protective ability of a protection means is particularly important.

In a further embodiment of the invention, the evaluation wavelength comprises wavelengths outside the wavelength range of 400 nm to 500 nm. In particular, the wavelength range of UVA and UVB is irradiated. This wavelength range poses the greatest risk to human skin, thus determining the protective ability of a protection means is particularly important.

In a further embodiment of the invention, the evaluation wavelength comprises wavelengths λ with λ<400 nm. To record an in vivo measurement, the UVA wavelength range (320 nm-400 nm) is evaluated in particular.

In a further embodiment of the invention, the evaluation wavelength comprises wavelengths λ with 320 nm<λ<400 nm. To record an in vivo measurement to determine the protective ability of the protection means, the UVA wavelength range (320 nm-400 nm) is evaluated in particular. To determine the protective ability of the protection means in the UVB wavelength range (<320 nm), this wavelength range can also be optionally evaluated.

In a further embodiment, the radiation is emitted from a single beam source, wherein the emitted radiation from the one beam source comprises light in a wavelength range between 280 nm and 500 nm. In the context of this document, a beam source is a technical device for generating electromagnetic radiation. A beam source is therefore not an optical element for guiding, deflecting or changing the intensity and/or wavelength of electromagnetic radiation. A beam source is therefore not, for example, a light guide, grating, prism, or filter. The beam source generates electromagnetic radiation in a wavelength range within the blue spectral range (from approximately 400 nm to 500 nm wavelength) and the UV range (280 nm to 400 nm). In a further development of the invention, the emitted radiation of the one beam source comprises only a partial range of the wavelength range between 280 nm and 500 nm, in a preferred embodiment the wavelength range of the radiation emitted by the one beam source is less than 50 nm wide and in a particularly preferred embodiment the one beam source is a single LED.

In a further development of the invention, the individual beam sources can be controlled. This enables targeted control of the beam source and targeted adaptation of the overall spectrum to different applications. By appropriately selecting the intensity of the beam source, the radiation dose is also achieved, either in the form of an individual dose (e.g. 0.1 MED) or an adjustable limit value. This minimizes the radiation dose for a test subject.

In a further aspect of the invention, the beam source is controlled by a beam source controller. The beam source control specifically controls the beam source and enables targeted adjustment of the spectrum generated by the beam source, e.g. by controlling the wavelength range and the intensity of the electromagnetic radiation generated by the beam source.

In a further embodiment of the invention, the wavelength, the exposure time and/or the intensity of the individual beam sources are controlled by controlling the beam source. This enables targeted control of the beam source for in vivo measurements on the one hand and in vitro measurements on the other. By appropriately selecting the intensity of the beam source and the exposure time of the measuring body, a radiation dose is also selected, either in the form of an individual dose (e.g. 0.1 MED) or an adjustable limit value. This minimizes the radiation dose for a test subject.

In a further aspect of the invention, the transmission spectrum is adjusted using the detected diffusely reflected radiation. The adjustment is carried out in such a way that a hybrid transmission spectrum T_{hyb} is determined according to:

$${T}_{hyb} ( λ ) = \text{?} ( λ ) * \text{?}$$

$$\text{?} \text{indicates text missing or illegible when filed}$$

with T_{in vivo} as the transmission spectrum determined from the spectrum of the detected diffusely reflected radiation determined by in vivo measurement and T_{in silico} the calculated transmission spectrum

In a further embodiment of the invention, the radiation is emitted in vivo on human skin in accordance with ISO 24442. ISO 24442 defines an in vivo method in which the UVA protection factor is determined using the minimum UVA dose required to produce irreversible pigmentation (suntan) of the skin.

In a further advantageous embodiment of the invention, the protective ability of the protection means is evaluated from two measurements. The protective ability of the protection means is measured by the time it takes for UVB rays to penetrate a protection means and cause the skin to redden (minimal erythema, MED), in a second measurement, compared to the time it takes to do so when no protection means is present, in a first measurement, the dose of solar radiation required to cause skin reddening is divided by the dose required to cause reddening without a protection means.

In a further development of the invention, a first measurement is carried out before the protection means is applied to the measuring body. This first measurement records the time at which minimal erythema (MED) appears on the test subject's untreated skin.

In a further aspect of the invention, a second measurement is carried out after the protection means has been applied to the measuring body. This second measurement records the time at which minimal erythema (MED) appears on the test subject's skin which has been treated with the protection means.

In a further embodiment of the invention, the beam source generates polychromatic radiation, wherein the generated polychromatic radiation is radiated unfiltered onto the measuring body. The beam source generates radiation in a maximum wavelength range of 280 nm to 500 nm (UVB to blue light). The generated polychromatic radiation is not altered by optical elements (filters, monochromators) in the generated wavelength range from the generation of the polychromatic radiation until it hits a measuring body. This results in a maximum intensity of the generated radiation, a likewise maximum intensity of the diffusely reflected or transmitted radiation and consequently a high signal-to-noise ratio.

In a further development of the invention, the measurement of the remission spectrum and/or remission value is carried out exclusively in vivo. In a first measurement, the remission spectrum and/or the remission value of the human skin without protection means is recorded, and in a second measurement, the remission spectrum and/or the remission value of the human skin without protection means is recorded.

The object is further achieved with the SPF evaluation system for the assessment of the protection factor of protection means according to claim 25. Further advantageous embodiments of the invention are also set forth in the dependent claims.

The SPF evaluation system according to the invention for the assessment of the protection factor of protection means comprises a measuring device, wherein the measuring device comprises a beam source device. The beam source device comprises a beam source, a detector unit, a control unit for controlling the measuring device and an evaluation unit.

The measuring device is suitable for introducing electromagnetic radiation into a measuring body, preferably the human skin, by means of the beam source. A beam source is a technical device for generating electromagnetic radiation.

In addition, the measuring device is suitable for detecting the diffusely reflected electromagnetic radiation. The measuring device can be controlled by means of the control unit, and the radiation detected by the detector unit can also be processed by means of the control unit. The detection window and the resolution of the spectrometer are determined by the control unit, the detected signals are stored, processed (e.g. amplified) and displayed and evaluated by the evaluation unit.

In a further development of the invention, the SPF evaluation system is suitable for the assessment of the protective ability of protection means for protection against light in an evaluation wavelength range. The evaluation wavelength range is the wavelength range for which the protection factor is determined. The evaluation wavelength range is different from the wavelength range of the irradiation wavelength and/or the detection wavelength, wherein the wavelength range of the evaluation wavelength range comprises at least the wavelength range of the irradiation wavelength and the detection wavelength.

In an advantageous embodiment of the invention, the SPF evaluation system has exactly one measuring device. In a further development of the invention, the exactly one measuring device has exactly one beam source device. In a further aspect of the invention, the exactly one beam source device comprises exactly one beam source. According to the invention, the measurement and detection of the electromagnetic radiation is not carried out with a sum spectrum generated by different beam sources, but with only exactly one measuring device, exactly one beam source device and exactly one beam source, wherein the beam source is preferably an LED. This makes the SPF evaluation system according to the invention significantly more compact and cost-effective than known systems.

In a further embodiment of the invention, the exactly one beam source device can be controlled by exactly one control unit for controlling the measuring device. The control unit can control the range of the irradiation wavelength and the intensity of the radiation emitted by the beam source device.

In a further embodiment of the invention, the exactly one measuring device has exactly one detector unit. In a further development of the invention, the exactly one detector unit has exactly one detector. In a further embodiment of the invention, the exactly one detector unit can be controlled by the exactly one control unit for controlling the measuring device. The detector unit includes, for example, a monochromator, filter, photomultiplier, spectrometer and/or a photodiode. All of these known devices are suitable for detecting electromagnetic radiation and measuring its intensity depending on the wavelength. The detection of an in vivo measurement is preferably carried out with a photodiode. This makes the SPF evaluation system according to the invention significantly more compact and cost-effective than known systems. The detection window and the resolution of the spectrometer are set by the control unit, the detected signals are stored, processed (e.g. amplified) and displayed by the control unit.

In a further embodiment of the invention, the exactly one beam source device is suitable for emitting light at an irradiation wavelength, wherein the wavelength range of the irradiation wavelength is smaller than the wavelength range of the evaluation wavelength. The exactly one beam source device generates electromagnetic radiation at an irradiation wavelength between the blue spectral range (from approximately 400 nm to 500 nm wavelength) and the UV range (280 nm to 400 nm). The evaluation wavelength is the wavelength for which the protection factor is determined. The evaluation wavelength is different from the wavelength range of the irradiation wavelength. The evaluation wavelength is like the irradiation wavelength and preferably a wavelength range, wherein the wavelength range of the evaluation wavelength comprises at least the wavelength range of the irradiation wavelength.

In a further embodiment of the invention, the exactly one detector unit is suitable for detecting light at a detection wavelength, wherein the wavelength range of the detection wavelength is smaller than the wavelength range of the evaluation wavelength. The detection wavelength, like the irradiation wavelength, preferably comprises a wavelength range, wherein the wavelength range of the detection wavelength preferably lies within the range of the irradiation wavelength or comprises the range of the irradiation wavelength.

In a further embodiment of the invention, the wavelength range of the irradiation wavelength or the wavelength range of the detection wavelength in the wavelength range of the evaluation wavelength is smaller than the wavelength range of the evaluation wavelength. To record an in vivo measurement, the wavelength range of the irradiation wavelength includes in particular the UVA wavelength range (320 nm-400 nm), which is irradiated into the measuring body. The wavelength range of the detection wavelength covers a maximum of the wavelength range of the irradiation wavelength (320 nm-400 nm). The wavelength range of the evaluation wavelength covers a wider range, in particular the wavelength range from 280 nm to 500 nm (UVB to blue light) is evaluated.

In a further development of the invention, the wavelength range of the irradiation wavelength and/or the wavelength range of the detection wavelength is less than 100 nm, preferably less than 50 nm and particularly preferably less than 25 nm. Therefore, only one steel source is needed to emit electromagnetic radiation, which has a narrow wavelength range of the irradiation wavelength. Similarly, to record the remission spectrum, a detector is required that can detect a small wavelength range of the detection wavelength. The beam source and the detector can therefore be implemented so that they can be manufactured and operated cost-effectively.

In a further embodiment of the invention, the SPF evaluation system has exactly one control unit. The control unit is usually a PC or notebook computer with a suitable computer program. By means of the control unit, the SPF evaluation system can be controlled; in addition, the radiation detected by the detector unit can be processed by the control unit. The wavelength range of the detection wavelength and the resolution of the detector unit are determined by means of the control device, the detected signals are stored, processed (e.g. amplified) and displayed. This enables targeted control of the beam source and targeted adaptation of the overall spectrum to different applications. By appropriately selecting the intensity of the beam source, the radiation dose is also achieved, either in the form of an individual dose (e.g. 0.1 MED) or an adjustable limit value. This minimizes the radiation dose for a test subject.

In an advantageous embodiment of the invention, the SPF evaluation system is suitable for the assessment of the protective ability of protection means for protection against light and/or for evaluating the protection factor of a protection means using measurement data exclusively from the measurement data device. The evaluation is carried out by means of the inventive method for determining a protection factor according to claim 1 to claim 24. By means of the SPF evaluation system, the diffusely reflected radiation of the irradiated measuring body (the human skin without and with protection means) can be detected. The evaluation of the protection means is carried out using the data of the remission spectrum and taking into account the data of a transmission spectrum, wherein according to the invention the data of the transmission spectrum are in silico data.

Exemplary embodiments of the method according to the invention for determining a protection factor, comprising a spectroscopic measurement and the SPF evaluation system according to the invention for the assessment of the protection factor of protection means are shown schematically in simplified form in the drawings and are explained in more detail in the following description.

In the drawings:

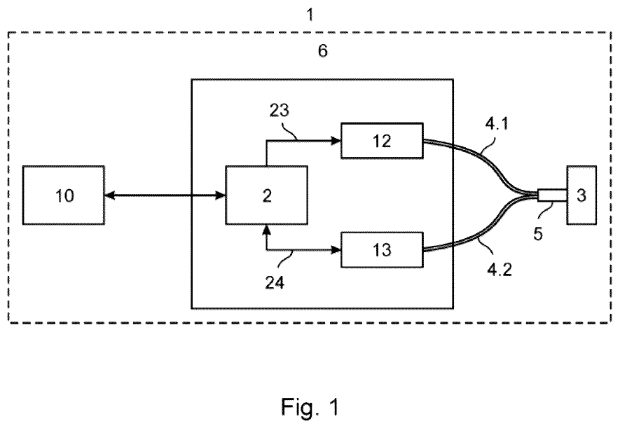

FIG. 1: SPF evaluation system, evaluation unit arranged outside the measuring device

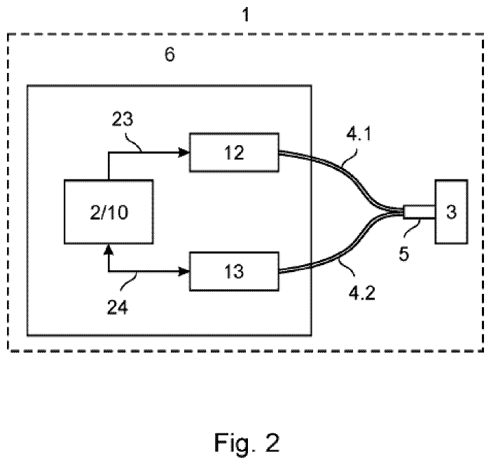

FIG. 2: SPF evaluation system, control unit and evaluation unit arranged in one device

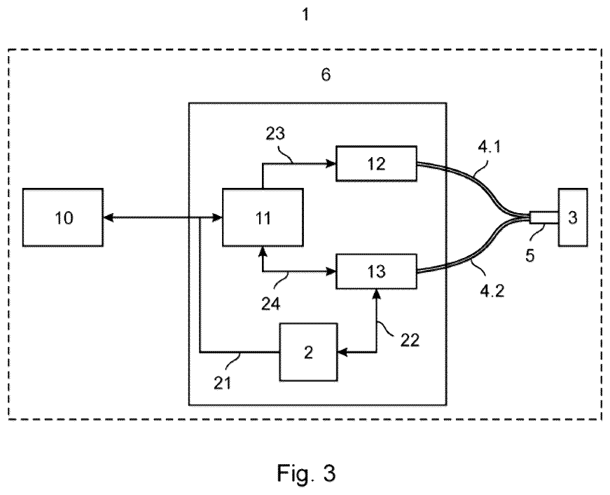

FIG. 3: SPF evaluation system, beam source control unit controlled by control unit

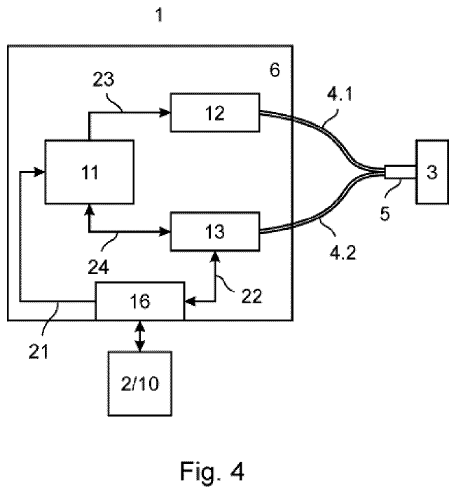

FIG. 4: SPF evaluation system, control unit and evaluation unit arranged separately in one device

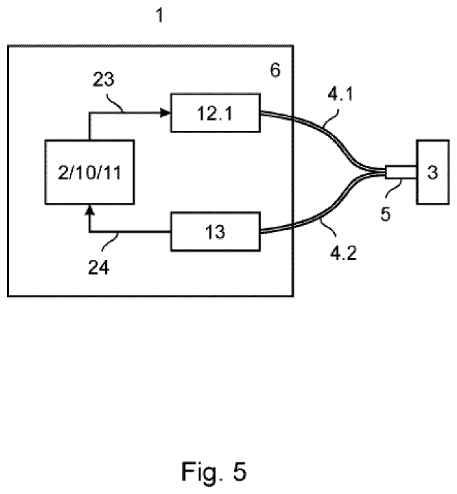

FIG. 5: SPF evaluation system, control unit, beam source control unit and evaluation unit arranged in one device

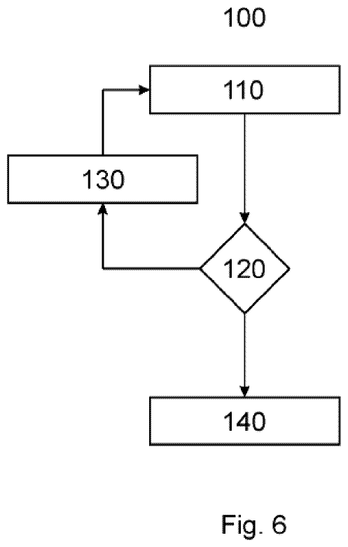

FIG. 6: Method for performing a spectroscopic measurement (in vivo) to record a reflection spectrum

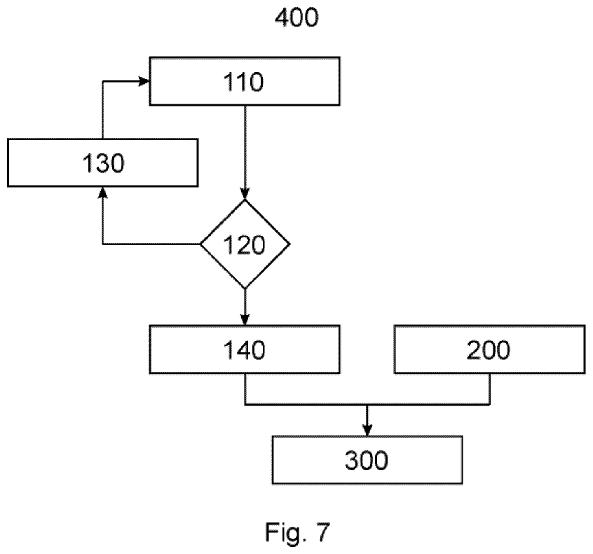

FIG. 7: Combined method for determining a protection factor with a measurement to record a reflection spectrum and in silico data in the form of a transmission spectrum

FIG. 1 schematically shows an embodiment of the SPF evaluation system 1 according to the invention for carrying out an in vivo measurement. The SPF evaluation system 1 comprises the measuring device 6 with a beam source device 12. The beam source device 12 comprises a beam source 12.1 and optical elements which are intended and/or suitable for conditioning and/or redirecting the radiation generated by the beam source 12.1, e.g. light guides, filters, monochromators, mirrors and/or other optical elements.

By means of a light guide 4.1, the light emitted by the beam source device 12 at an irradiation wavelength between 280 nm and 500 nm is introduced into the measuring body 3 via the probe head 5. The light reflected by the measuring body 3 reaches the detector unit 13 via a further light guide 4.2. The detector unit 13 has a monochromator, filter, photomultiplier, spectrometer and/or a photodiode. In this and all subsequent embodiments, the detector unit 13 comprises a photodiode. Detector unit 13 and beam source device 12 are connected via data lines 23, 24 to a control unit 2, which in turn is connected to the evaluation unit 10 via a further data line 25. The control unit 2 is usually a PC or notebook computer with a suitable computer program. Control unit 2 and detector unit 13 are also connected to each other via a data line 22.

A further embodiment of the SPF evaluation system 1 according to the invention is shown in FIG. 2. The SPF evaluation system 1 also has the measuring device 6 with a beam source device 12. By means of the light guide 4.1, the light emitted by the beam source device 12 is introduced into the measuring body 3 via the probe head 5, and the light reflected by the measuring body 3 reaches the detector unit 13 via a further light guide 4.2. The detector unit 13 and the beam source device 12 are connected via data lines 23, 24 to the control unit 2, which in this embodiment forms a structural unit with the evaluation unit 10.

A further embodiment of the SPF evaluation system 1 according to the invention also for carrying out an in vivo measurement is shown in FIG. 3. The SPF evaluation system 1 has the measuring device 6 with a beam source device 12. By means of the light guide 4.1, the light emitted by the beam source device 12 is introduced into the measuring body 3 via the probe head 5, and the light reflected by the measuring body 3 reaches the detector unit 13 via a further light guide 4.2. The detector unit 13 and the beam source device 12 are connected via data lines 23, 24 to a beam source control 11. Beam source control 11 and detector unit 13 are each connected to the control unit 2 via the data line 21, the evaluation unit 10 is arranged separately and is also connected to the control unit 2 and the beam source control 11 via the data line 21.

A further embodiment of the SPF evaluation system 1 according to the invention is shown in FIG. 4. The SPF evaluation system 1 also has the beam source device 12. By means of the light guide 4.1, the light emitted by the beam source device 12 is introduced into the measuring body 3 via the probe head 5, and the light reflected by the measuring body 3 reaches the detector unit 13 via a further light guide 4.2. The detector unit 13 and the beam source device 12 are connected via data lines 23, 24 to a beam source control 11. In this embodiment, the control unit 2 is arranged in a structural unit with the evaluation unit remote from the SPF evaluation system 1 and is connected to it via the interface 16. The connection can be wired or wireless, e.g., via IP connection, Bluetooth, etc. Interface 16 and detector unit 13 on the one hand and interface 16 and beam source control 11 are connected to each other via data lines 21, 22.

A preferred embodiment of the SPF evaluation system 1 according to the invention, also for carrying out an in vivo measurement, is shown in FIG. 5. The SPF evaluation system 1 corresponds to the system presented in the first embodiment (see FIG. 1), namely SPF evaluation system 1, only the control unit 2 is arranged in a structural unit with the beam source control 11 and the evaluation unit 10 in the measuring device 6. The SPF evaluation system 1 has exactly one measuring device 6 with exactly one beam source 12.1 and exactly one detector unit 13. Beam source 12.1 is an LED, the detector unit 13 is a photodiode. The exactly one control unit 2 in a structural unit with the exactly one evaluation unit 10 and the exactly one beam source control 11 controls the exactly one beam source 12.1 via the data line 23 and receives data from the exactly one detector unit 13 via the data line 24. This SPF evaluation system 1 presented here has a particularly compact design, requires only a small number of components and is therefore cost-effective to manufacture and use.

FIG. 6 shows an embodiment of an implementation of the method 100 of an in vivo measurement for detecting the remission spectrum by means of the SPF evaluation system 1 according to the invention from the previous embodiments (see. FIG. 1 to FIG. 5). The test is carried out in vivo according to ISO 24442 or 24444. The method 100 of a measurement requires the recording of a remission spectrum of the skin of test subject 3 untreated with a protection means and of the skin treated with a protection means.

For this purpose, the probe head 5 is applied to the untreated skin of the test subject 3, i.e. the protection means to be tested is not applied to the skin of the test subject 3. For this purpose, a location on the inner side of the forearm or the back of a test subject 3 is usually selected. The first measurement 110 is then carried out by the beam source control 11 controlling the LED 12.1 in such a way that the light emitted by the LED 12.1 (see FIG. 4) is guided through the light guide 4.1 onto the skin of the test subject 3.

The generated electromagnetic radiation has an irradiation wavelength range with a FWHM of up to 20 nm, with the generated electromagnetic radiation being in the wavelength range from 330 nm to 350 nm. The wavelength range of the detection wavelength in which the detector unit 13 detects the diffusely reflected radiation by means of the light guide 4.2 also has a range of, for example, 20 nm in the range from 330 nm to 350 nm. Moreover, the range of the irradiation wavelength and/or the wavelength range of the detection wavelength is smaller than 100 nm, preferably smaller than 50 nm and particularly preferably smaller than 25 nm

For some applications, FWHM can be up to 30 nm, with the generated electromagnetic radiation in the wavelength range of 400 nm to 800 nm. For other applications, FWHMs of up to 100 nm or more can be used and/or reduced by filters if necessary, with the generated electromagnetic radiation in the wavelength range from 800 nm to 2000 nm.

The light generated by the LED 12.1 is irradiated unfiltered onto the measuring body 3 to ensure a high S/N ratio. In particular, the light generated by the LED 12.1 is polychromatic with an intensity maximum at a wavelength in the UVA range of 340 nm. Alternatively, an LED can be used that produces light with a maximum intensity in the UVA range of 365 nm. The irradiation occurs at an intensity that does not cause acute damage to the skin, which is below the simple MED, or below the MPE [maximum permissible exposure] values, or significantly below the values caused by solar radiation. The light diffusely reflected by the skin of the test subject 3 is guided through the light guide 4.2 to the photodiode of the detector unit 13, detected by the photodiode and converted into measured values, the measured values are sent to the control unit 2 and stored in the control unit 2.

The control unit 2 then asks from 120 whether the second measurement of the skin of test subject 3 treated with protection means has already been carried out. If this is not the case, control unit 2 will indicate this. To carry out the second measurement 110 with applied protection means, the protection means is applied 130 to the skin of test subject 3, e.g. according to ISO 24442 or 24444 in the amount of 2.0 mg/cm² on the skin surface to be tested. The application 130 of the protection means and the subsequent second measurement 110 are carried out at the same location of the measurement sample 3, in particular on the same location of the skin of a test subject, in order to ensure the reproducibility of the first and second measurements 110. Also to ensure reproducibility, the control unit 2 controls the beam source control 11 in such a manner that the beam source control 11 controls the LED 12.1 so that the light generated by the LED 12.1 is guided through the light guide 4.1 onto the skin of the test subject 3, wherein the intensity and/or exposure time of the first and second measurement 110 are coordinated with one another.

The light diffusely reflected by the skin of the test subject 3 is also detected by the photodiode of the detector unit 13 and converted into measured values. The measured values are sent to control unit 2 and stored in control unit 2.

If the query 120 shows that the second measurement 110 has already been carried out, an evaluation 140 of the protection means is carried out. For this purpose, the control unit 2 executes a program to calculate the reflection spectrum T_{in vivo} according to equation 1:

$${T}_{in vivo} = \frac{1}{{SPF}_{in vivo}} = \sqrt{\frac{R}{{R}_{0}}} \qquad Eq . 1$$

with T_{in vivo} as a function of the wavelength λ, SPF_{in vivo} the protection factor determined by the in vivo method 100, R₀ the reflected intensity of the untreated skin of test subject 3 as a function of the wavelength λ, R the reflected intensity of the skin of test subject 3 treated with protection means as a function of the wavelength λ. The method 100 presented here requires a time expenditure of a few seconds to a few tens of seconds.

As an alternative to the method shown in FIG. 6 with one LED, this method can also be carried out in the same way with a multi-LED radiation source.

FIG. 7 shows an embodiment of the method 400 according to the invention for carrying out a combined in vivo reflection measurement 100 (see FIG. 5) including the data of a transmission spectrum 200. According to the invention, the values of the transmission spectrum 200 are calculated (in silico).

If the amounts and properties of the UV filter substances of a protection means are known, the UV transmission can be calculated, taking into account the irregularity of the film and its photodegradation. Based on the simulated UV transmission, the SPF and all parameters characterizing protection against UVA and/or UVB can be calculated in silico.

The in silico determination of the data of a transmission spectrum 200 is carried out according to ISO 24443. An application of 2.0 g/cm² of protection means is assumed. The emitted radiation is assumed to be in the wavelength range from 280 nm to 500 nm (UVB to blue light), so the wavelength range includes the wavelength range of the evaluation wavelength from a maximum of 280 nm to 500 nm.

The results of the evaluations of the in vivo remission spectrum 140 and the in silico transmission spectrum 200 are evaluated together 300. For this purpose, the hybrid transmission spectrum T_{hyb} is first calculated, where the in silico transmission spectrum T_{in silico} is scaled by the reflection spectrum T_{in vivo}:

The two results of the individual evaluations 140, 230 are combined and evaluated 300. For this purpose, the hybrid transmission spectrum T_{hyb} is calculated, where the in vitro transmissions spectrum T_{in silico} is scaled by the reflection spectrum T_{in vivo}

$${T}_{hyb} ( λ ) = {T}_{in silico} ( λ ) * \frac{{T}_{in vivo} ( {λ}_{LED} )}{{T}_{in silico} ( {λ}_{LED} )} \qquad Eq . 3$$

The protective ability of the protection means for the spectral range from UVA (320 nm) to the HEV spectral range (450 nm) SF is then calculated according to equation 4 (here E=IPD(λ) is the IPD spectrum; S=I(λ) is the solar spectrum):

$$S F = \frac{{\int }_{320}^{450} S ( λ ) E ( λ ) d λ}{{\int }_{320}^{450} S ( λ ) E ( λ ) {T}_{hyb} d λ} \qquad Eq . 4$$

The protective ability of the protection means in the spectral range of blue light (400 nm to 500 nm) of interest for the present invention is determined according to equation 5 (here E=IPD(λ) is the IPD spectrum; S=I(λ) is the solar spectrum)

$$SF ( 400 nm - 500 nm ) = \frac{{\int }_{400}^{500} S ( λ ) E ( λ ) d λ}{{\int }_{400}^{500} S ( λ ) E ( λ ) {T}_{hyb} d λ} \qquad Eq . 5$$

The protective ability of the protection means from 400 nm to 450 nm SF (400 nm-450 nm) is determined according to equation 6 (here E=IPD(λ) is the IPD spectrum; S=I(λ) is the solar spectrum):

$$SF ( 400 nm - 450 nm ) = \frac{{\int }_{400}^{450} S ( λ ) E ( λ ) d λ}{{\int }_{400}^{450} S ( λ ) E ( λ ) {T}_{hyb} d λ} \qquad Eq . 6$$

The protective ability of the protection means for the spectral range of UVA (320-400 nm) UVA-SF is then calculated according to equation 4 (here E=PPD(λ) is the PPD spectrum; S=I(λ) is the solar spectrum or UVA source for PPD-test.):

$$UVA - SF = \frac{{\int }_{320}^{400} S ( λ ) E ( λ ) d λ}{{\int }_{320}^{400} S ( λ ) E ( λ ) {T}_{hyb} d λ} \qquad Eq . 7$$

The protective ability of the protection means for the spectral range from UVB to UVA (280-400 nm) UV-SF is then given by equation 4

$$U V - S F = \frac{{\int }_{290}^{400} S ( λ ) E ( λ ) d λ}{{\int }_{290}^{400} S ( λ ) E ( λ ) {T}_{h y b} d λ} \qquad Eq . 8$$

In all equations for SF or UVA-SR, an optional correction function F (SF)=SF_korr or F (UVA-SF)=UVA-PF_korr is used, which describes, for example, skin type-dependent differences. A correction function makes values of different skin types comparable and outputs a corrected value SFₖₒᵣᵣ or UVA-PFₖₒᵣᵣ.

For example, F can be a linear factor (i.e., F(SF)=SF*C) or an exponential function (i.e., F(SF)=SF^{c}). C can depend, for example, on the skin type or the ITA° value. As shown in Eq. 4, the formulas in Eqs. 2 to 8 can be written as follows:

$$\text{?} = F ( SF ) = F ( \frac{{\int }_{320}^{450} S ( λ ) E ( λ ) d λ}{{\int }_{320}^{450} S ( λ ) E ( λ ) {T}_{hyb} d λ} ) \qquad Eq . 9$$

$$\text{?} \text{indicates text missing or illegible when filed}$$

Since the in vivo value is measured without photodegradation, photodegradation should be appropriately taken into account. This can be done by calculating T_in silico with T_{in silico_irr}(λ) and without photodegradation and calculating a spectral quotient from it

$$SRPD ( λ ) = \frac{{T}_{in silico \_ irr} ( λ )}{{T}_{in silico} ( λ )} . \qquad Eq . 10$$

With this

$${T}_{hyb \_ irr} ( λ ) = {T}_{hyb} , ( λ ) * S R P D ( λ ) \qquad Eq . 11$$

is then calculated

The method can be calibrated using suitable reference methods such as electron spin resonance spectroscopy.

### LIST OF REFERENCE NUMERALS

1 SPF evaluation system
2 Control device
3 Sample/measuring body
4.1, 4.2 Light guide/fiber bundle
5 Probe head
6 Measuring device
10 Evaluation device
11 Beam source control
12 Beam source device
12.1 Beam source
13 Detection unit
21 Connection beam source control—control device
22 Connection detector/spectrometer—control device
23 Connection beam source control—beam source device
24 Connection beam source control—detection unit
25 Connection control device—evaluation device
100 Method for recording a spectroscopic measurement (in vivo)
110 Performing a reflection spectrum measurement
120 Query
130 Applying the protection means
140 Evaluation of the reflection spectrum
200 Method for determining the data of a transmission spectrum
300 Determination of a protection factor
400 Method for determining a protection factor

1. A method (400) for determining a protection factor, comprising the steps of:
emitting radiation from a beam source (12.1), wherein the emitted radiation comprises light at an irradiation wavelength between 280 nm and 2000 nm, preferably 280 to 800 nm, particularly preferably the range from 280 to 500 nm,
irradiating a measuring body (3) using the emitted radiation,
detecting the radiation diffusely reflected by the irradiated measuring body (3) at a detection wavelength,
evaluating the protection factor of a protection means using an evaluation wavelength from the diffusely reflected radiation and a transmission spectrum
wherein the data of a transmission spectrum are used to evaluate the protection factor of the protection means,
wherein the evaluation wavelength is different from the wavelength range of the irradiation wavelength and/or the detection wavelength, and wherein the transmission spectrum data are in silico data.

2. The method (400) for determining a protection factor according to claim 1,
characterized in that
the wavelength range of the transmission spectrum includes the evaluation wavelength.

3. The method (400) for determining a protection factor according to claim 1,
characterized in that
the evaluation wavelength covers the wavelength range from 280 nm to 2000 nm, preferably the wavelength range from 280 nm to 800 nm or particularly preferably the wavelength range from 280 nm to 500 nm and/or the wavelength range from 400 nm to 500 nm and/or the wavelength range from 400 nm to 450 nm.

4. The method (400) for determining a protection factor according to claim 3,
characterized in that
the evaluation of the protective ability of the protection means for light in a wavelength range from 400 nm to 500 nm is carried out in a separate process from the evaluation of the protective ability of the protection means for light in a wavelength range from 280 nm to 400 nm.

5. The method (400) for determining a protection factor according to claim 4,
characterized in that
the evaluation of the protective ability of the protection means for light in a wavelength range from 400 nm to 500 nm is carried out in a separate process from the evaluation of the protective ability of the protection means for light in a wavelength range from 280 nm to 400 nm.

6. The method (400) for determining a protection factor according to claim 1,
characterized in that
the wavelength range of the irradiation wavelength or the wavelength range of the detection wavelength is smaller than the wavelength range of the evaluation wavelength.

7. The method (400) for determining a protection factor according to claim 6,
characterized in that
the wavelength range of the irradiation wavelength or the wavelength range of the detection wavelength in the wavelength range of the evaluation wavelength is smaller than the wavelength range of the evaluation wavelength.

8. The method (400) for determining a protection factor according to claim 1,
characterized in that
the range of the irradiation wavelength and/or the wavelength range of the detection wavelength is smaller than 100 nm, preferably smaller than 50 nm and particularly preferably smaller than 25 nm.

9. The method (400) for determining a protection factor according to claim 1,
characterized in that
the irradiation wavelength and/or the detection wavelength includes only light with wavelengths outside the wavelength range of 400 nm to 450 nm.

10. The method (400) for determining a protection factor according to claim 1,
characterized in that
the irradiation wavelength and/or the detection wavelength includes only light with wavelengths outside the wavelength range of 400 nm to 500 nm.

11. The method (400) for determining a protection factor according to claim 1,
characterized in that
the evaluation wavelength includes wavelengths outside the wavelength range of 400 nm to 500 nm.

12. The method (400) for determining a protection factor according to claim 11,
characterized in that
the evaluation wavelength includes wavelengths λ with λ<400 nm.

13. The method (400) for determining a protection factor according to claim 12,
characterized in that
the evaluation wavelength includes wavelengths λ with 320 nm<λ<400 nm.

14. The method (400) for determining a protection factor according to claim 1,
characterized in that
the emission of radiation occurs from a single beam source (12.1), wherein the irradiation wavelength of the one beam source (12.1) comprises light in a wavelength range between 280 nm and 500 nm.

15. The method (400) for determining a protection factor according to claim 14,
characterized in that
the individual beam source (12.1) can be controlled.

16. The method (400) for determining a protection factor according to claim 14,
characterized in that
the control of the beam source (12.1) is controlled by a beam source control (11).

17. The method (400) for determining a protection factor according to claim 16,
characterized in that
by controlling the beam source (12.1) the wavelength, the exposure time and/or the intensity of the individual beam sources (12.1) are controlled.

18. The method (400) for determining a protection factor according to claim 1,
characterized in that
the transmission spectrum is adjusted using the detected diffusely reflected radiation.

19. The method (400) for determining a protection factor according to claim 1,
characterized in that
the radiation is emitted in vivo onto human skin.

20. The method (400) for determining a protection factor according to claim 1,
characterized in that
the evaluation of the protective ability of the protection means is carried out from two measurements.

21. The method (400) for determining a protection factor according to claim 20,
characterized in that
a first measurement is taken before the protection means is applied to the measuring body (3).

22. The method (400) for determining a protection factor according to claim 20,
characterized in that
a second measurement is carried out after the application (130) of the protection means to the measuring body (3).

23. The method (400) for determining a protection factor according to claim 1,
characterized in that
the beam source (12.1) generates polychromatic radiation, wherein the generated polychromatic radiation is radiated unfiltered onto the measuring body (3).

24. The method (400) for determining a protection factor according to claim 1,
characterized in that
the spectroscopic measurement (100) is carried out exclusively in vivo.

25. An SPF evaluation system (1) for the assessment of the protection factor of protective products, comprising the following components:
a measuring device (6) with
a beam source device (12),
wherein the beam source device (12) comprises a beam source (12.1),
a detector unit (13),
a control unit (2) for controlling the measuring device (6),
an evaluation unit (10).

26. The SPF evaluation system (1) for the assessment of the protection factor of protection means according to claim 25,
characterized in that
the SPF evaluation system (1) is suitable for the assessment of the protective ability of protection means for protection against light in an evaluation wavelength range.

27. The SPF evaluation system (1) for the assessment of the protection factor of protection means according to claim 25,
characterized in that
the SPF evaluation system (1) has exactly one measuring device (6).

28. The SPF evaluation system (1) for the assessment of the protection factor of protection means according to claim 27,
characterized in that
the exactly one measuring device (6) has exactly one beam source device (12).

29. The SPF evaluation system (1) for the assessment of the protection factor of protection means according to claim 28,
characterized in that
the exactly one beam source device (12) has exactly one beam source (12.1).

30. The SPF evaluation system (1) for the assessment of the protection factor of protection means according to claim 28,
characterized in that
the exactly one beam source device (12) can be controlled by exactly one control unit (2) for controlling the measuring device (6).

31. The SPF evaluation system (1) for the assessment of the protection factor of protection means according to claim 27,
characterized in that
the exactly one measuring device (6) has exactly one detection unit (13).

32. The SPF evaluation system (1) for the assessment of the protection factor of protection means according to claim 31,
characterized in that
the exactly one detection unit (13) has exactly one detector.

33. The SPF evaluation system (1) for the assessment of the protection factor of protection means according to claim 31,
characterized in that
the exactly one detection unit (13) can be controlled by exactly one control unit (2) for controlling the measuring device (6).

34. The SPF evaluation system (1) for the assessment of the protection factor of protection means according to claim 25,
characterized in that
the exactly one beam source device (12) is suitable for emitting light at an irradiation wavelength,
wherein the wavelength range of the irradiation wavelength is smaller than the wavelength range of the evaluation wavelength.

35. The SPF evaluation system (1) for the assessment of the protection factor of protection means according to claim 25,
characterized in that
the exactly one detection unit (13) is suitable for detecting light at a detection wavelength,
wherein the wavelength range of the irradiation wavelength is smaller than the wavelength range of the evaluation wavelength.

36. The SPF evaluation system (1) for the assessment of the protection factor of protection means according to claim 34,
characterized in that
the wavelength range of the irradiation wavelength or the wavelength range of the detection wavelength in the wavelength range of the evaluation wavelength is smaller than the wavelength range of the evaluation wavelength.

37. The SPF evaluation system (1) for the assessment of the protection factor of protection means according to claim 34,
characterized in that
the range of the irradiation wavelength and/or the wavelength range of the detection wavelength is smaller than 100 nm, preferably smaller than 50 nm and particularly preferably smaller than 25 nm.

38. The SPF evaluation system (1) for the assessment of the protection factor of protection means according to claim 25,
characterized in that
the SPF evaluation system (1) has exactly one control unit (2).

39. The SPF evaluation system (1) for the assessment of the protection factor of protection means according to claim 25,
characterized in that
the SPF evaluation system (1) is suitable for the assessment of the protective ability of protection means for protection against light and/or the protection factor of a protection means using exclusively measuring data from a measuring data device (6).
